(19) United States
(12) Patent Application Publication
(10) Pub. No.: US 2004/0146961 A1
(43) Pub. Date: Jul. 29, 2004

(54) Fret-based homogeneous in vitro phosphate transfer assay system

(76) Inventor: Brian Noland, San Diego, CA (US)

Correspondence Address: Sheryl Silverstein, Structural GenomiX, Inc., 10505 Roselle Street, San Diego, CA, 92121, US

(21) Appl. No.: 10/738,231

(22) Filed: Dec. 17, 2003

Related U.S. Application Data
(60) Provisional application No. 60/435,458, filed on Dec. 20, 2002.

(51) Int. Cl. C12Q 1/48; C12Q 1/42

(52) U.S. Cl. 435/21; 435/15

(57) ABSTRACT

The invention relates to a novel method of assaying phosphate transfer, such as that mediated by kinases or phosphatases, using fluorescence resonance energy transfer. These assays may be used, for example, for high throughput screening of enzymes having phosphate transfer activity.

### INTRODUCTION

This application claims benefit of priority from U.S. Provisional Patent Application 60/435,458, filed Dec. 20, 2002, which is hereby incorporated by reference as if fully set forth.

The invention relates to a novel method of assaying phosphate transfer, such as that mediated by kinases or phosphatases, using fluorescence resonance energy transfer. These assays may be used, for example, for high throughput screening of enzymes having phosphate transfer activity, and, for example, to screen compounds to determine the phosphorylation state, or the phosphorylatability.

### BACKGROUND OF THE INVENTION

Many cellular processes are regulated by phosphate transfer, including phosphate transfer mediated by kinases or phosphatases. Kinases phosphorylate, that is, add phosphate groups to, compounds. Phosphatases dephosphorylate, that is, remove phosphate groups from, compounds. Compounds that may be phosphorylated or dephosphorylated include, for example, proteins, peptides, lipids, sugars, and small molecules. Kinases and phosphatases that have protein substrates have been implicated in important cellular processes, for example, signal transduction, cell division, and initiation of gene transcription. Kinases and phosphatases have been considered to be good targets for drug therapy, including therapy for cancer.

There is a need for a simple, efficient, sensitive, assay for phosphate transfer, that is well suited for both traditional and high throughput screening.

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

The present invention presents a simple, efficient, sensitive, assay for phosphate transfer. Embodiments of the present invention include, but are not limited to, the following:

Embodiment 1. A method of determining the phosphorylating activity of an enzyme comprising the steps of:

a. Combining said enzyme with:

i. a phosphorylatable compound, wherein said compound is labeled with an acceptor fluorophore label; and

ii. an ATP analog comprising a reactive species on the γ-phosphate group of said ATP analog;

b. Combining the product of step (a) with a donor fluorophore label, wherein

i. said donor fluorophore label corresponds to the acceptor fluorophore labeling said compound; and wherein

ii. said donor fluorophore label comprises an alkylating moiety that is capable of specifically modifying said reactive species;

c. Measuring the fluorescence resonance energy transfer of the combination of steps (a) and (b); and

d. Using the fluorescence resonance energy transfer of step (c) to determine the phosphorylating activity of the enzyme.

Embodiment 2. The method of embodiment 1, wherein said enzyme is a kinase.

Embodiment 3. The method of embodiment 1, wherein said compound is a peptide.

Embodiment 4. The method of embodiment 3, wherein said peptide comprises an amino acid selected from the group consisting of serine, threonine and tyrosine, and wherein said peptide is capable of being phosphorylated at said amino acid by said enzyme to yield a product.

Embodiment 5. A method of determining the phosphorylating activity of an enzyme comprising the steps of:

a. Combining said enzyme with:

i. a peptide comprising an amino acid selected from the group consisting of serine, threonine and tyrosine, wherein said peptide is capable of being phosphorylated at said amino acid by said enzyme to yield a product, and wherein said peptide is labeled with an acceptor fluorophore label; and

ii. an ATP analog comprising a reactive species on the γ-phosphate group of said ATP analog;

b. Combining the product of step (a) with a donor fluorophore label, wherein

i. said donor fluorophore label corresponds to the acceptor fluorophore labeling said peptide; and wherein

ii. said donor fluorophore label comprises an alkylating moiety that is capable of specifically modifying said reactive species;

c. Measuring the fluorescence resonance energy transfer of the combination of steps (a) and (b); and

d. Using the fluorescence resonance energy transfer of step (c) to determine the phosphorylating activity of the enzyme.

Embodiment 6. A method of determining the phosphorylating activity of an enzyme comprising the steps of:

a. Combining said enzyme with:

i. a phosphorylatable compound, wherein said compound is labeled with a donor fluorophore label; and

ii. an ATP analog comprising a reactive species on the γ-phosphate group of said ATP analog;

b. Combining the product of step (a) with an acceptor fluorophore label, wherein

i. said acceptor fluorophore label corresponds to the donor fluorophore labeling said compound; and wherein

ii. said acceptor fluorophore label comprises an alkylating moiety that is capable of specifically modifying said reactive species;

c. Measuring the fluorescence resonance energy transfer of the combination of steps (a) and (b); and

d. Using the fluorescence resonance energy transfer of step (c) to determine the phosphorylating activity of the enzyme.

Embodiment 7. The method of embodiment 6, wherein said enzyme is a kinase.

Embodiment 8. The method of embodiment 6, wherein said compound is a peptide. Embodiment 9. The method of embodiment 8, wherein said peptide comprises an amino acid selected from the group consisting of serine, threonine and tyrosine, and wherein said peptide is capable of being phosphorylated at said amino acid by said enzyme to yield a product.

Embodiment 10. A method of determining the phosphorylating activity of an enzyme comprising the steps of:

a. Combining said enzyme with:

i. a peptide comprising an amino acid selected from the group consisting of serine, threonine and tyrosine, wherein said peptide is capable of being phosphorylated at said amino acid by said enzyme to yield a product, and wherein said peptide is labeled with a donor fluorophore label; and

ii. an ATP analog comprising a reactive species on the γ-phosphate group of said ATP analog;

b. Combining the product of step (a) with an acceptor fluorophore label, wherein

i. said acceptor fluorophore label corresponds to the donor fluorophore labeling said peptide; and wherein

ii. said acceptor fluorophore label comprises an alkylating moiety that is capable of specifically modifying said reactive species;

c. Measuring the fluorescence resonance energy transfer of the combination of steps (a) and (b); and

d. Using the fluorescence resonance energy transfer of step (c) to determine the phosphorylating activity of the enzyme.

Embodiment 11. A method of determining the phosphorylation of a compound by an enzyme, comprising the steps of:

a. Combining said enzyme with:

i. the compound, wherein said compound is labeled with an acceptor fluorophore label; and

ii. an ATP analog comprising a reactive species on the γ-phosphate group of said ATP analog;

b. Combining the product of step (a) with a donor fluorophore label, where

i. said donor fluorophore label corresponds to the acceptor fluorophore labeling said compound; and wherein

c. said donor fluorophore label comprises an alkylating moiety that is capable of specifically modifying said reactive species;

i. measuring the fluorescence resonance energy transfer of the combination of steps (a) and (b); and

ii. using the fluorescence resonance energy transfer of step (c) to determine whether the compound has been phosphorylated.

Embodiment 12. The method of embodiment 11, wherein said enzyme is a kinase.

Embodiment 13. The method of embodiment 11, wherein said compound is a peptide.

Embodiment 14. The method of embodiment 13, wherein said peptide comprises an amino acid selected from the group consisting of serine, threonine and tyrosine, and wherein said peptide is capable of being phosphorylated at said amino acid by said enzyme to yield a product.

Embodiment 15. A method of determining the phosphorylation of a compound by an enzyme, comprising the steps of:

a. Combining said enzyme with:

i. the compound, wherein said compound is labeled with a donor fluorophore label; and

b. An ATP analog comprising a reactive species on the γ-phosphate group of said ATP analog;

c. Combining the product of step (a) with an acceptor fluorophore label, wherein

i. said acceptor fluorophore label corresponds to the donor fluorophore labeling said compound, and wherein

ii. said acceptor fluorophore label comprises an alkylating moiety that is capable of specifically modifying said reactive species;

d. Measuring the fluorescence resonance energy transfer of the combination of steps (a) and (b); and

e. Using the fluorescence resonance energy transfer of step (c) to determine whether the compound has been phosphorylated.

Embodiment 16. The method of embodiment 15, wherein said enzyme is a kinase.

Embodiment 17. The method of embodiment 15, wherein said compound is a peptide.

Embodiment 18. The method of embodiment 17, wherein said peptide comprises an amino acid selected from the group consisting of serine, threonine and tyrosine, and wherein said peptide is capable of being phosphorylated at said amino acid by said enzyme to yield a product.

Embodiment 19. A method of determining the dephosphorylating activity of an enzyme, comprising the steps of:

a. Combining said enzyme with:

i. a compound, wherein

1. said compound comprises a phosphate group, wherein said phosphate group comprises a reactive species, and wherein

2. said compound is labeled with an acceptor fluorophore label; and

ii. a donor fluorophore label, wherein

1. said donor fluorophore label corresponds to the acceptor fluorophore labeling said compound; and wherein

2. said donor fluorophore label comprises an alkylating moiety that is capable of specifically modifying said reactive species;

b. Measuring the fluorescence resonance energy transfer of the combination of step (a); and

c. Using the fluorescence resonance energy transfer of step (b) to determine the dephosphorylating activity of the test enzyme.

Embodiment 20. The method of embodiment 19, wherein said enzyme is a kinase.

Embodiment 21. The method of embodiment 19, wherein said compound is a peptide. Embodiment 22. The method of embodiment 21, wherein said peptide comprises an amino acid selected from the group consisting of serine, threonine and tyrosine, and wherein said peptide is capable of being phosphorylated at said amino acid by said enzyme to yield a product.

Embodiment 23. A method of determining the dephosphorylating activity of an enzyme, comprising the steps of:

a. Combining said enzyme with:

i. a peptide, wherein

1. said peptide comprises an amino acid selected from the group consisting of serine, threonine and tyrosine, wherein said amino acid is phosphorylated with a phosphate group comprising a reactive species, and wherein

2. said peptide is labeled with an acceptor fluorophore label; and

ii. a donor fluorophore label, wherein

1. said donor fluorophore label corresponds to the acceptor fluorophore labeling said peptide, and wherein

2. said donor fluorophore label comprises an alkylating moiety that is capable of specifically modifying said reactive species;

b. Measuring the fluorescence resonance energy transfer of the combination of step (a); and

c. Using the fluorescence resonance energy transfer of step (b) to determine the dephosphorylating activity of the test enzyme.

Embodiment 24. A method of determining the dephosphorylating activity of an enzyme, comprising the steps of:

a. Combining said enzyme with:

i. a compound, wherein

1. said compound comprises a phosphate group, wherein said phosphate group comprises a reactive species, and wherein

2. said compound is labeled with a donor fluorophore label; and

ii. an acceptor fluorophore label, wherein

1. said acceptor fluorophore label corresponds to the donor fluorophore labeling said compound, and wherein

2. said acceptor fluorophore label comprises an alkylating moiety that is capable of specifically modifying said reactive species;

b. Measuring the fluorescence resonance energy transfer of the combination of step (a); and

c. Using the fluorescence resonance energy transfer of step (b) to determine the dephosphorylating activity of the test enzyme.

Embodiment 25. The method of embodiment 24, wherein said enzyme is a kinase.

Embodiment 26. The method of embodiment 24, wherein said compound is a peptide.

Embodiment 27. The method of embodiment 26, wherein said peptide comprises an amino acid selected from the group consisting of serine, threonine and tyrosine, and wherein said peptide is capable of being phosphorylated at said amino acid by said enzyme to yield a product.

Embodiment 28. A method of determining the dephosphorylating activity of an enzyme, comprising the steps of:

a. Combining said enzyme with:

i. a peptide, wherein

1. said peptide comprises an amino acid selected from the group consisting of serine, threonine and tyrosine, wherein said amino acid is phosphorylated with a phosphate group comprising a reactive species,

2. and wherein said peptide is labeled with a donor fluorophore label; and

ii. an acceptor fluorophore label, wherein

1. said acceptor fluorophore label corresponds to the donor fluorophore labeling said peptide, and wherein

2. said acceptor fluorophore label comprises an alkylating moiety that is capable of specifically modifying said reactive species;

b. Measuring the fluorescence resonance energy transfer of the combination of step (a); and

c. Using the fluorescence resonance energy transfer of step (b) to determine the dephosphorylating activity of the test enzyme.

Embodiment 29. A method of determining the dephosphorylation of a compound by an enzyme, comprising the steps of:

a. Combining said enzyme with:

i. the compound, wherein

1. said compound comprises a phosphate group, wherein said phosphate group comprises a reactive species, and wherein

2. said compound is labeled with an acceptor fluorophore label; and

ii. a donor fluorophore label, wherein

1. said donor fluorophore label corresponds to the acceptor fluorophore labeling said compound, and wherein

2. said donor fluorophore label comprises an alkylating moiety that is capable of specifically modifying said reactive species;

b. Measuring the fluorescence resonance energy transfer of the combination of step (a); and

c. Using the fluorescence resonance energy transfer of step (b) to determine whether the compound has been dephosphorylated.

Embodiment 30. The method of embodiment 29, wherein said enzyme is a kinase.

Embodiment 31. The method of embodiment 29, wherein said compound is a peptide.

Embodiment 32. The method of embodiment 31, wherein said peptide comprises an amino acid selected from the group consisting of serine, threonine and tyrosine, and wherein said peptide is capable of being phosphorylated at said amino acid by said enzyme to yield a product.

Embodiment 33. A method of determining the dephosphorylation of a compound by an enzyme, comprising the steps of:

a. Combining said enzyme with:

i. the compound, wherein

1. said compound comprises a phosphate group, wherein said phosphate group comprises a reactive species, and wherein

2. said compound is labeled with a donor fluorophore label; and

ii. an acceptor fluorophore label, wherein

1. said acceptor fluorophore label corresponds to the donor fluorophore labeling said compound, and wherein

2. said acceptor fluorophore label comprises an alkylating moiety that is capable of specifically modifying said reactive species;

b. Measuring the fluorescence resonance energy transfer of the combination of step (a); and

c. Using the fluorescence resonance energy transfer of step (b) to determine whether the compound has been dephosphorylated.

Embodiment 34. The method of embodiment 33, wherein said enzyme is a kinase.

Embodiment 35. The method of embodiment 33, wherein said compound is a peptide.

Embodiment 36. The method of embodiment 35, wherein said peptide comprises an amino acid selected from the group consisting of serine, threonine and tyrosine, and wherein said peptide is capable of being phosphorylated at said amino acid by said enzyme to yield a product.

Embodiment 37. The method of any of embodiments 1-18, wherein said ATP analog comprises ATP-γS.

Embodiment 38. The method of any of embodiments 19-36, wherein said reactive species is sulfur.

Embodiment 39. The method of any of embodiments 1-5, 11-14, 19-23, and 29-32, wherein said acceptor fluorophore label comprises fluorescein, and said donor fluorophore label comprising an alkylating moiety is 1,5-IAEDANS.

Embodiment 40. The method of any of embodiments 3-5, 13, 14, 21-23, 31, and 32, wherein

a. Said peptide comprises EAIYAAPFAKKK, comprising said acceptor label at Lys12;

b. Said acceptor fluorophore label comprises fluorescein, and wherein

c. Said donor fluorophore label comprising an alkylating moiety is 1,5-IAEDANS.

Embodiment 41. The method of any of embodiments 6-10, 15-18, 24-28, and 33-36, wherein said donor fluorophore label comprises carboxytetramethylrhodamine, and said acceptor fluorophore label comprising an alkylating moiety is QSY-7 maleimide.

Embodiment 42. The method of any of embodiments 8-10, 17, 18, 26-28, 35, and 36, wherein

a. said peptide comprises EAIYAAPFAKKK, comprising said donor label at Lys12

b. said donor fluorophore label comprises carboxytetramethylrhodamine, and wherein

c. said acceptor fluorophore label comprising an alkylating moiety is QSY-7 maleimide.

Embodiment 43. An assay system or kit, comprising the following reagents

a. A phosphorylatable compound, wherein said compound is labeled with an acceptor fluorophore label;

b. An ATP analog comprising a reactive species on the γ-phosphate group of said ATP analog;

c. A donor fluorophore label, wherein

i. said donor fluorophore label corresponds to the acceptor fluorophore labeling said compound; and wherein

ii. said donor fluorophore label comprises an alkylating moiety that is capable of specifically modifying said reactive species.

Embodiment 44. An assay system or kit, comprising the following reagents

a. A phosphorylatable compound, wherein said compound is labeled with a donor fluorophore label;

b. An ATP analog comprising a reactive species on the γ-phosphate group of said ATP analog;

c. An acceptor fluorophore label, wherein

i. Said acceptor fluorophore label corresponds to the donor fluorophore labeling said compound; and wherein

ii. said acceptor fluorophore label comprises an alkylating moiety that is capable of specifically modifying said reactive species.

Embodiment 45. The assay system of embodiment 43 or 44, wherein said compound is a peptide.

Embodiment 46. The assay system of embodiment 45, wherein said peptide comprises an amino acid selected from the group consisting of serine, threonine and tyrosine, and wherein said peptide is capable of being phosphorylated at said amino acid by said enzyme to yield a product.

Embodiment 47. The assay system of embodiment 43, wherein said acceptor fluorophore label comprises fluorescein, and said donor fluorophore label comprising an alkylating moiety is 1,5-IAEDANS.

Embodiment 48. The assay system of embodiment 47, wherein said compound is a peptide.

Embodiment 49. The assay system of embodiment 48, wherein said peptide comprises an amino acid selected from the group consisting of serine, threonine and tyrosine, and wherein said peptide is capable of being phosphorylated at said amino acid by said enzyme to yield a product.

Embodiment 50. The assay system of embodiment 49, wherein said peptide comprises EAIYAAPFAKKK, comprising said acceptor label at Lys12;

Embodiment 51. The assay system of embodiment 44, wherein said donor fluorophore label comprises carboxytetramethylrhodamine, and said acceptor fluorophore label comprising an alkylating moiety is QSY-7 maleimide.

Embodiment 52. The assay system of embodiment 51, wherein said compound is a peptide.

Embodiment 53. The assay system of embodiment 52, wherein said peptide comprises an amino acid selected from the group consisting of serine, threonine and tyrosine, and wherein said peptide is capable of being phosphorylated at said amino acid by said enzyme to yield a product.

Embodiment 54. The assay system of embodiment 53, wherein said peptide comprises EAIYAAPFAKKK, comprising said donor label at Lys12

Embodiment 55. The assay system of embodiment 43 or 44, wherein each of said reagents is in a separate container.

Embodiment 56. The assay system of embodiment 55, wherein said containers are enclosed in a package, which package further includes instructions for use of said reagents.

Embodiment 57. The assay system of embodiment 43 or 44, further comprising a microtray.

Embodiment 58. The method of embodiment 43 or 44, wherein said ATP analog comprises ATP-γS.

Embodiment 59. An assay system or kit, comprising the following reagents

a. A compound, wherein

i. Said compound comprises a phosphate group, wherein said phosphate group comprises a reactive species, and wherein

ii. said compound is labeled with an acceptor fluorophore label;

b. A donor fluorophore label, wherein

i. said donor fluorophore label corresponds to the acceptor fluorophore labeling said compound; and wherein

ii. said donor fluorophore label comprises an alkylating moiety that is capable of specifically modifying said reactive species.

Embodiment 60. An assay system comprising the following reagents

a. A compound, wherein

i. said compound comprises a phosphate group, wherein said phosphate group comprises a reactive species, and wherein

ii. said compound is labeled with a donor fluorophore label;

b. An acceptor fluorophore label, wherein

i. Said acceptor fluorophore label corresponds to the donor fluorophore labeling said compound; and wherein

ii. said acceptor fluorophore label comprises an alkylating moiety that is capable of specifically modifying said reactive species.

Embodiment 61. The assay system of embodiment 59 or 60, wherein said compound is a peptide.

Embodiment 62. The assay system of embodiment 61, wherein said peptide comprises an amino acid selected from the group consisting of serine, threonine and tyrosine, and wherein said peptide phosphorylated at said amino acid.

Embodiment 63. The assay system of embodiment 59, wherein said acceptor fluorophore label comprises fluorescein, and said donor fluorophore label comprising an alkylating moiety is 1,5-IAEDANS.

Embodiment 64. The assay system of embodiment 63, wherein said compound is a peptide.

Embodiment 65. The assay system of embodiment 64, wherein said peptide comprises an amino acid selected from the group consisting of serine, threonine and tyrosine, and wherein said peptide is phosphorylated at said amino acid.

Embodiment 66. The assay system of embodiment 65, wherein said peptide comprises EAIYAAPFAKKK, comprising said acceptor label at Lys12;

Embodiment 67. The assay system of embodiment 60, wherein said donor fluorophore label comprises carboxytetramethylrhodamine, and said acceptor fluorophore label comprising an alkylating moiety is QSY-7 maleimide.

Embodiment 68. The assay system of embodiment 67, wherein said compound is a peptide.

Embodiment 69. The assay system of embodiment 68, wherein said peptide comprises an amino acid selected from the group consisting of serine, threonine and tyrosine, and wherein said peptide is phosphorylated at said amino acid.

Embodiment 70. The assay system of embodiment 69, wherein said peptide comprises EAIYAAPFAKKK, comprising said donor label at Lys12

Embodiment 71. The assay system of embodiment 59 or 60, wherein each of said reagents is in a separate container.

Embodiment 72. The assay system of embodiment 71, wherein said containers are enclosed in a package, which package further includes instructions for use of said reagents.

Embodiment 73. The assay system of embodiment 59 or 60, further comprising a microtray.

Embodiment 74. The method of embodiments 59 or 60, wherein said reactive species is sulfur

The invention is illustrated by way of the present application, including working examples demonstrating embodiments of the assays.

### BRIEF DESCRIPTION OF THE FIGURES

FIG. 1 is a diagram of a FRET kinase assay. Step (1) is a kinase reaction where the protein kinase transfers a reactive phosphate species onto a peptide or protein substrate labeled with label 1. Step (2) is the alkylation step where the reactive phosphate group R is alkylated with an alkylating reagent specific to R. ATPγS is one example of a suitable reactive phosphate species because it is easily obtained commercially and it transfers a reactive thiol to the peptide. There are several commercially available thiol-reactive dyes that can serve as either donors or acceptors in the FRET assay system, for example, but not limited to, dyes with iodoacetimides or maleimides. Labels 1 and 2 are dyes that are appropriate donor-acceptor pairs having overlapping donor emission and acceptor absorbance spectra for FRET to occur in the final product.

FIG. 2 is a “Lights-On” FRET format emission spectra with 1,5-IAEDANS donor and fluorescein acceptor. The excitation wavelength was 325 nm and the emission envelope was measured from 400-640 nm. The pathlength was 1 cm and both readings were done at ambient temperature. The sample with enzyme added is shown in the dashed trace and the negative control with no enzyme added is shown in the solid trace.

FIGS. 3(A-I) is a “Lights-Out” FRET format emission spectra with TAMRA donor and QSY-7 maleimide acceptor. The excitation wavelength was 554 nm and the emission envelope was measured from 565-700 nm. The pathlength was 1 cm and both readings were done at ambient temperature. The sample with enzyme added is shown in the dashed trace and the negative control with no enzyme added is shown in the solid trace.

FIG. 4 is a coupled kinase-phosphatase assay diagram. The kinase reaction is the same as described in FIG. 1 with the exception of a phosphatase that may be added in the same reaction.

FIG. 5 depicts a schema for a discontinuous FRET protein kinase assay system.

FIG. 6 depicts a schema for an additional coupled kinase-phosphatase assay.

FIG. 7 depicts a time course of alkylation. The alkylation reaction is set up as described above. A. The resulting kinetic traces are fit by a mathematical model that describes a single first-order exponential decay. B. The resulting first order rate constants from the fits from panel A are graphed against concentration of QSY 7 malemide. The slope of the resulting line gives the second order rate constant for alkylation (100M⁻¹min⁻¹).

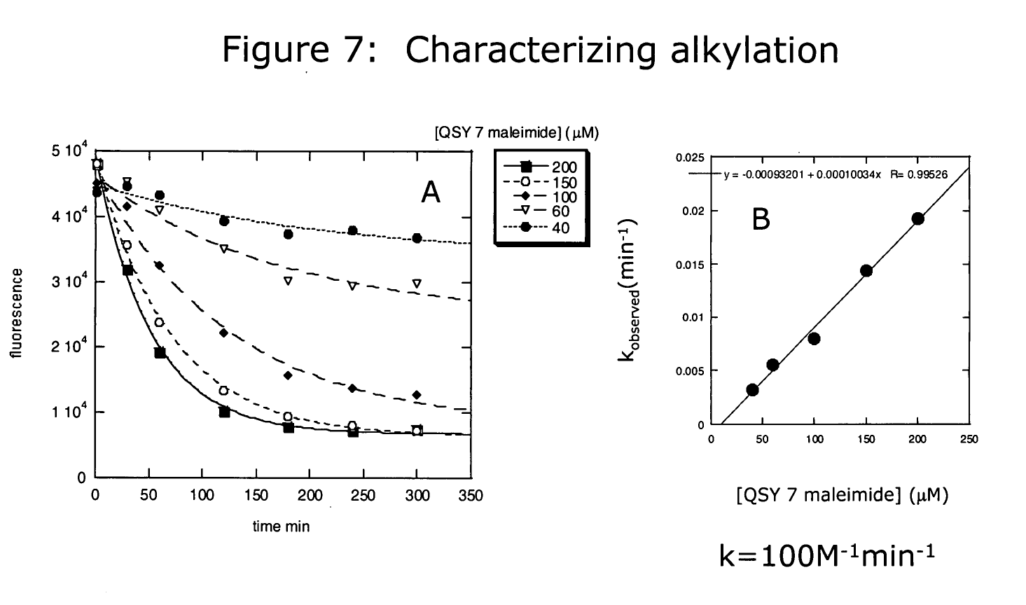

FIG. 8 is a bar graph of an example of a divalent metal salt screen in the “Lights-out” FRET assay for c-Abl. The reactions are set up as described. The change in fluorescence (i.e. the amount of positive signal) is determined by subtracting the positive sample from the appropriate negative control (supplemented with 50 mM EDTA).

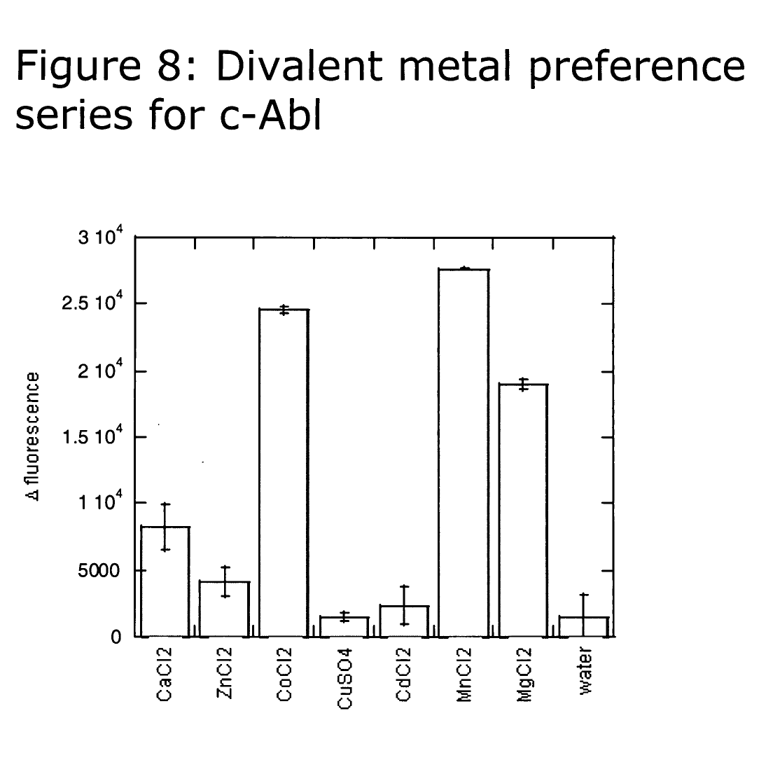

FIG. 9 depicts a time course of a kinase reaction for c-Abl. The divalent salt used was 0.1 mM CoCl₂, the peptide substrate is the CT-TAMRA Abl peptide at 5 μM, ATPγS is 10 μM and the enzyme concentration is indicated on the plot. Reactions are set up as described. Each kinetic trace is fit to a mathematical model that describes a single first-order exponential decay. Such time courses may be employed to determine the linear range for enzyme concentration in the assay.

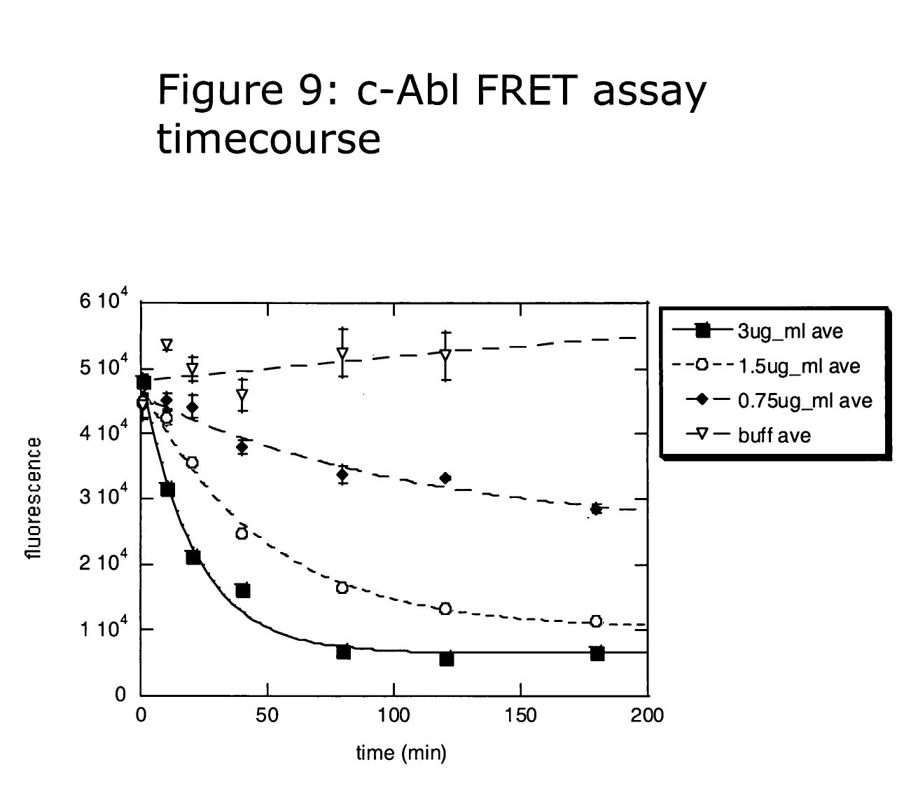

FIG. 10 depicts an example of “Lights-out” FRET assay for screening reference compounds against c-Abl. Reference inhibitor compound is added to every other well of a 384-well plate. Positive controls contain DMSO at 5%. Negative controls contain 50 mM EDTA. The Z′ score for this screen plate is calculated to be 0.861.

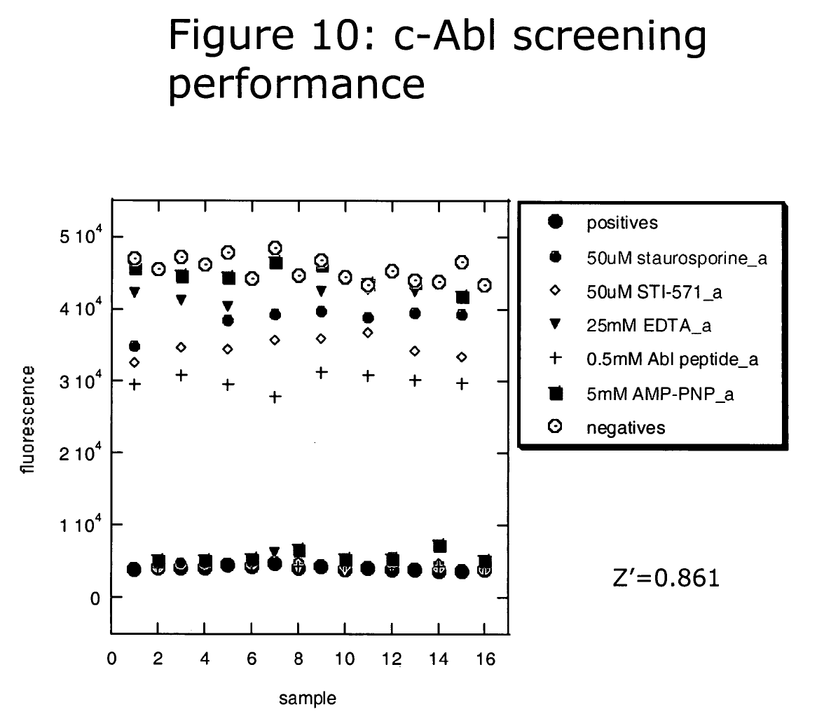

FIG. 11 depicts inhibitor titrations in the c-Alb FRET assay. Assays are set up and run as described. The test compounds staurosporine (stauro), Gleevec (ST1571), ATP and EDTA are all serially diluted in the assay. The resulting dose response curves are then analyzed by a competitive dose response equation.

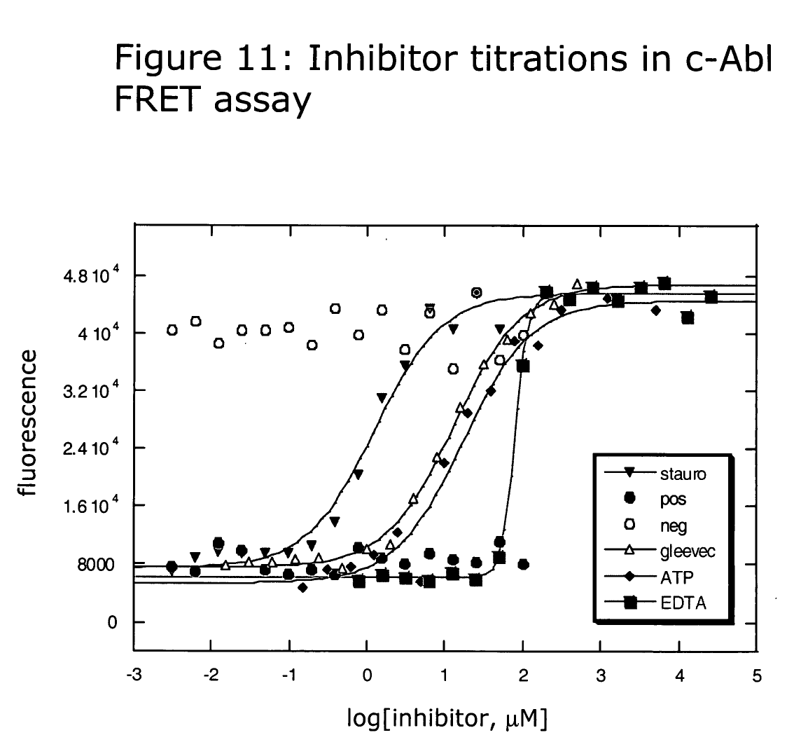

FIG. 12 depicts a time course of a kinase reaction for Syk. Reactions are set up as described. The divalent salt used was 1 mM CoCl₂, the peptide substrate is the NT-TAMRA Syk peptide at 5 μM, ATPγS is 20 μM and the enzyme concentration is indicated on the plot. Each kinetic trace is fit to a mathematical model that describes a single first-order exponential decay. Such time courses may not be employed to determine the linear range for enzyme concentration in the assay.

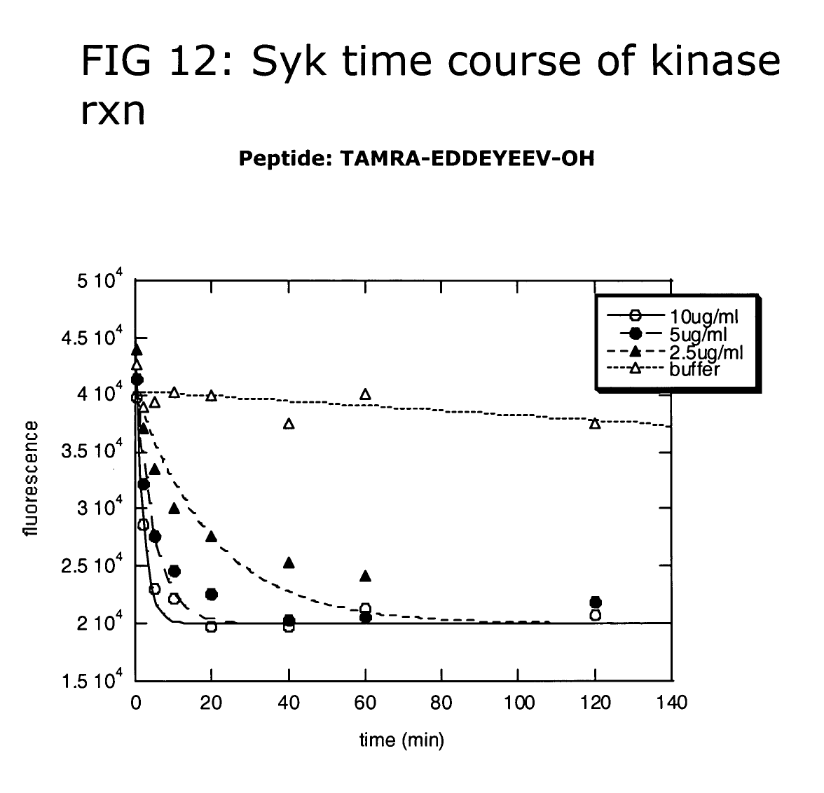

FIG. 13 depicts a time course of a kinase reaction for EGFR. Reactions are set up as described. The divalent salt used is 1 mM CoCl₂, the peptide substrate is the NT-TAMRA Syk peptide at 10 μM, ATPγS is 40 μM A and the enzyme concentration is indicated on the plot. Each kinetic trace is fit to a mathematical model that describes a single first-order exponential decay. Such time courses may be employed to determine the linear range for enzyme concentration in the assay.

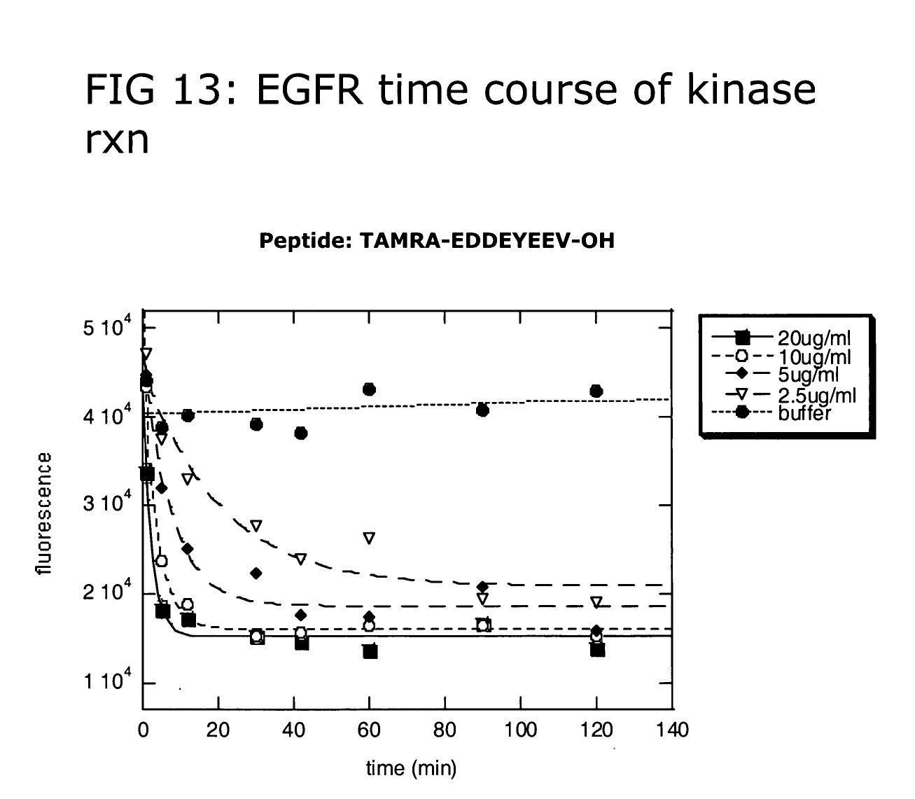

FIG. 14 depicts a time course of a kinase reaction for FES. Reactions are set up as described. The divalent salt used is 1 mM CoCl₂, the peptide substrate is the NT-TAMRA Syk peptide at 10 μM, ATPγS is 20 μM A and the enzyme concentration is indicated on the plot. Each kinetic trace is fit to a mathematical model that describes a single first-order exponential decay. Such time courses may be employed to determine the linear range for enzyme concentration in the assay.

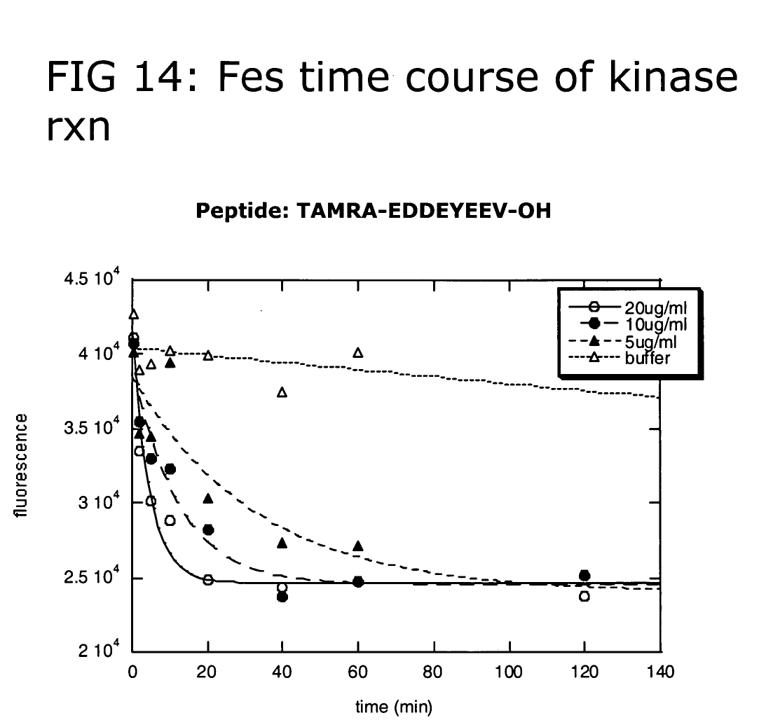

FIG. 15 depicts a time course of a kinase reaction for ARG. Reactions are set up as described. The divalent salt used is 0.1 mM CoCl₂, the peptide substrate is the CT-TAMRA Abl peptide at 5 μM, ATPγS is 10 μM A and the enzyme concentration is indicated on the plot. Each kinetic trace is fit to a mathematical model that describes a single first-order exponential decay. Such time courses may be employed to determine the linear range for enzyme concentration in the assay.

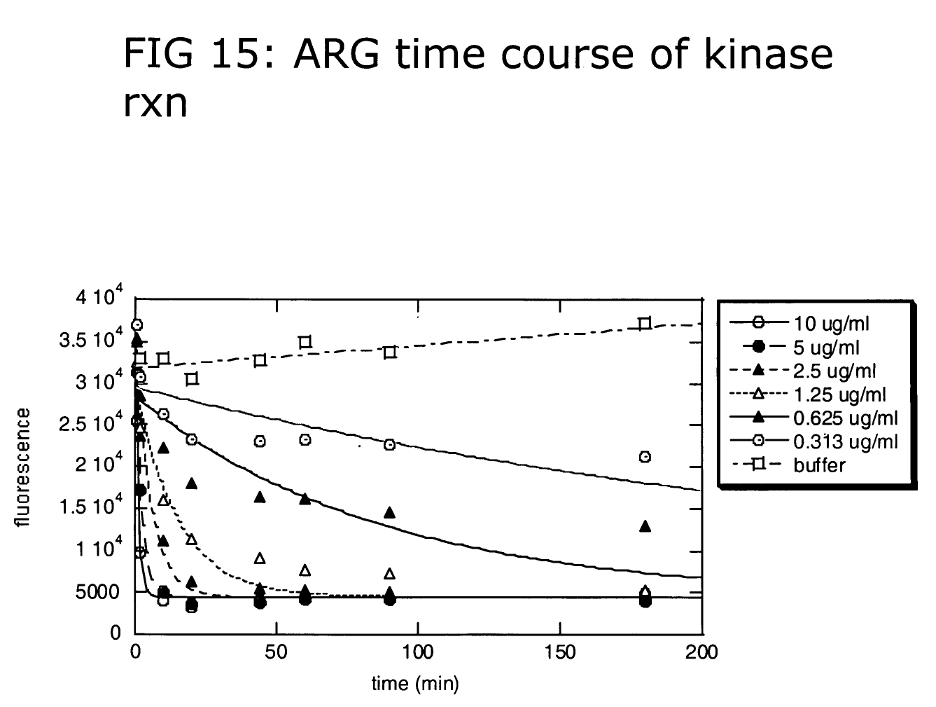

FIG. 16 is a bar graph depicting a divalent metal salt screen in the “Lights-out” FRET assay for PKA from bovine heart. The reactions are set up as described. The substrate peptide used is CT-EDANS Kemptide at 25 μM, ATPγS is 60 μM A and 100 U/mL PKA. The change in fluorescence (i.e. the amount of positive signal) is determined by subtracting the positive sample from the appropriate negative control (supplemented with 50 mM EDTA).

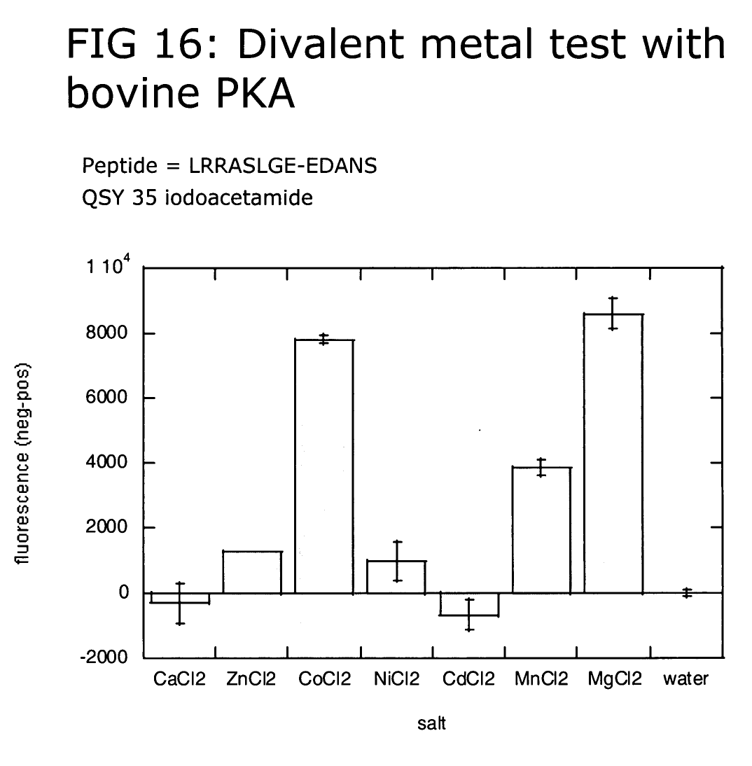

### ABBREVIATIONS

The amino acid notations used herein for the twenty genetically encoded amino acids are:

| | One-Letter | Three-Letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter amino acid abbreviations designate amino acids in the L-configuration. Amino acids in the D-configuration are preceded with a “D-.” For example, Arg designates L-arginine and D-Arg designates D-arginine. Likewise, the capital one-letter abbreviations refer to amino acids in the L-configuration. Lower-case one-letter abbreviations designate amino acids in the D-configuration. For example, “R” designates L-arginine and “r” designates D-arginine.

Unless noted otherwise, when polypeptide sequences are presented as a series of one-letter and/or three-letter abbreviations, the sequences are presented in the N→C direction, in accordance with common practice.

Definitions

By “or” is meant one, or another member of a group, or more than one member. For example, A, B, or C, may indicate any of the following: A alone; B alone; C alone; A and B; B and C; A and C; A, B, and C.

By “kinase” is meant an enzyme capable of phosphorylating a substrate. Where the substrate is a protein or peptide, the kinase is capable of phosphorylating the protein or peptide at a Ser, Thr, or Tyr residue.

By “phosphatase” is meant an enzyme capable of dephosphorylating a substrate. Where the substrate is a protein or peptide, the kinase is capable of dephosphorylating the protein or peptide at a phosphoserine, phosphothreonine, or phosphotyrosine residue.

By “substrate” is meant a molecule on which a kinases or phosphatase acts. Substrates are capable of being recipients of a phosphate or a donor of a phosphate, as mediated by a kinase or phosphatase. For protein kinases and protein phosphatases, the substrate is a protein or a peptide.

By “phosphorylatable compound” is meant that the compound is capable of being a recipient of a phosphate or a donor of a phosphate, as mediated by a kinase or phosphatase.

By “reactive species” is meant an atom or site of a molecule where covalent chemistry can occur.

By “phosphorylating activity” is meant kinase activity, and by “dephosphorylating activity” is meant phosphatase activity.

By “product” is meant the product (phosphorylated or dephosphorylated substrate) of a phosphate transfer reaction.

By “phosphate transfer” is meant the transfer of a phosphate onto or off of a substrate.

### DETAILED DESCRIPTION OF THE INVENTION

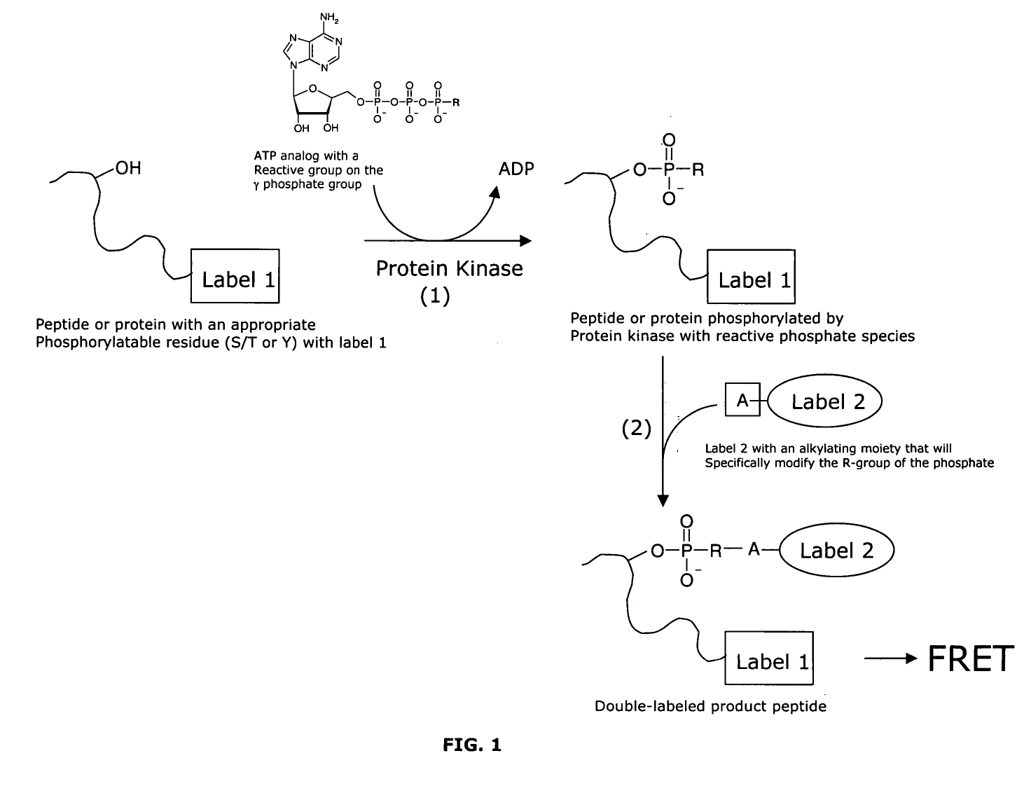

The invention relates to an in vitro phosphate transfer assay based on FRET (fluorescence resonance energy transfer) that is homogenous, simple, and adaptable to microtiter plate format. The phosphate may be transferred to and from a compound, such as, for example, a protein, a peptide, a lipid, a sugar, or a small molecule. A diagram of an embodiment of a kinase assay of the invention is shown in FIG. 1. In the first step, the reaction is done in the presence of a protein being tested for kinase activity, a control kinase, or a negative control, such as buffer; labeled peptide or protein substrate; and an ATP analog with a reactive species on the terminal phosphate. Enzymatic transfer of this terminal phosphate species results in a phosphopeptide product that contains the reactive species. In the next step the reactive species is alkylated with a second label (label 2) that contains an alkylating moiety that will specifically modify the R group on the reactive phosphate species. The quantity of double-labeled peptide is directly proportional to the activity of the protein kinase. Such an assay can be used to test the activity of protein kinases in the presence or absence of inhibitors. Using this assay in a format suitable for high-throughput screening such as, for example, in a microplate, flowcell, solid support, bead, or a microchip, should allow for high-throughput screening of inhibitors of protein kinases.

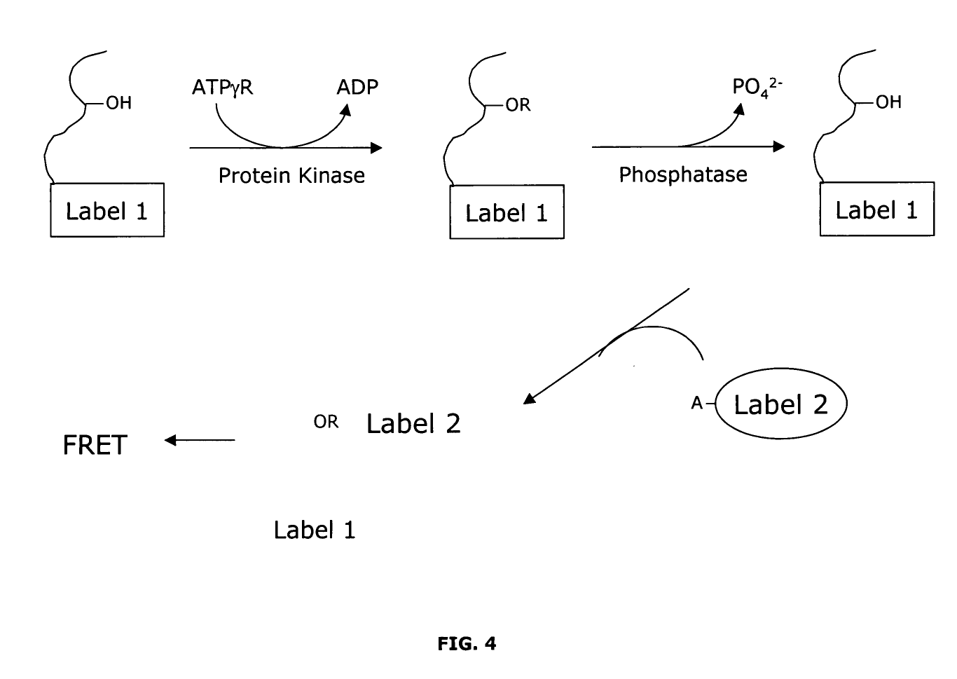

A diagram of an embodiment of a phosphatase assay of the invention is shown in FIG. 4. In the first step, a kinase catalyzed reaction is done in the presence of a labeled peptide or protein substrate and an ATP analog with a reactive species on the terminal phosphate. Enzymatic transfer of this terminal phosphate species results in a phosphopeptide product that contains the reactive species. A protein being tested for phosphatase activity, a positive phosphatase control, or a negative control, such as buffer, is included in the reaction, or may be added as a second step. In the next step, the reactive species is alkylated with a second label (label 2) that contains an alkylating moiety that will specifically modify the R group on the reactive phosphate species. The quantity of double-labeled peptide is inversely proportional to the activity of the protein phosphatase. Such an assay could be used to test the activity of protein phosphatases in the presence or absence of inhibitors. Using this assay in a format suitable for high-throughput screening such as, for example, in a microplate flowcell, solid support, bead, or a microchip, format should allow for high-throughput screening of inhibitors of protein kinases.

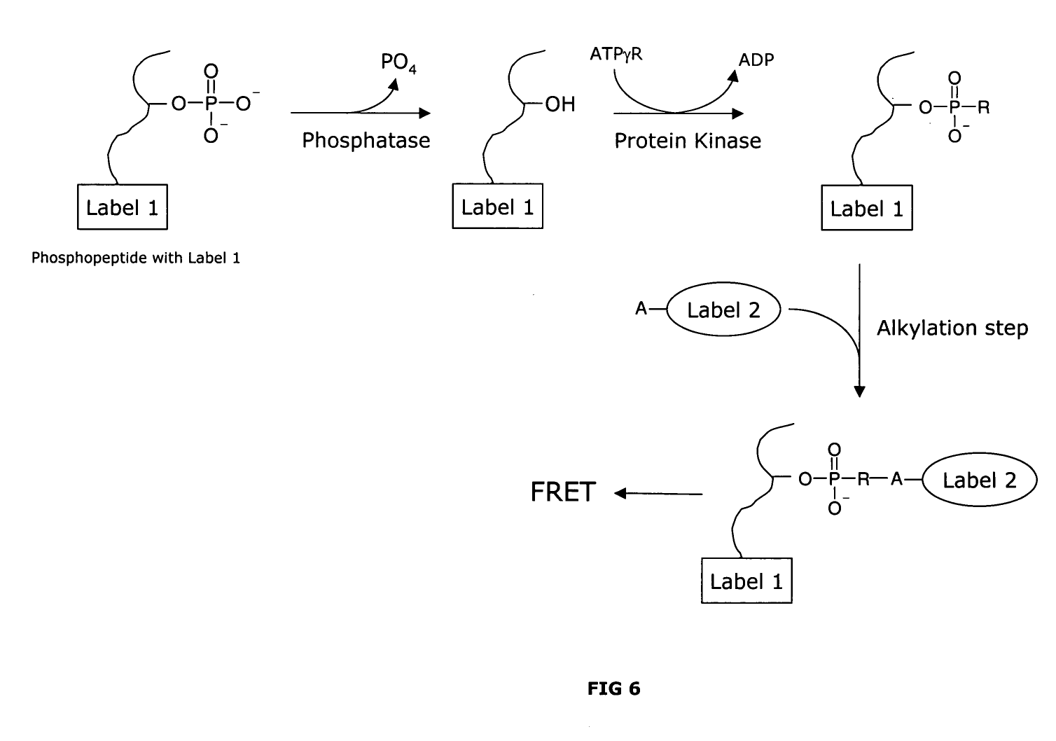

Certain reactive phosphate analogs may show increased resistance to dephosphorylation by phosphatases. Phosphorylation of proteins with ATP□S makes them resistant to dephosphorylation by protein phosphatases (Cassel, D., Glaser, L. (1982) Proc. Natl. Acad. Sci. U.S.A. Apr; 79(7):2231-2235). Shown in FIG. 6 is another phosphatase coupled FRET assay format that uses a phosphopeptide labeled with label 1 and eliminates the potential problem of phosphatase resistant modification. In this format, the phosphatase dephosphorylates a labeled phosphopeptide to yield a peptide with a free hydroxyl at the position occupied by the phosphate. In a subsequent step, a protein kinase phosphorylates this hydroxyl with an ATP analog with a reactive phosphate species on the □phosphate. This reactive species is then specifically alkylated with label 2. The amount of double-labeled peptide is then measured by FRET. In this format, the amount of double-labeled peptide is proportional to the activity of the phosphatase. Using this assay in a format suitable for high-throughput screening, for example, in a microplate flowcell, solid support, bead, or a microchip format should allow for high-throughput screening of inhibitors of protein phosphatases.

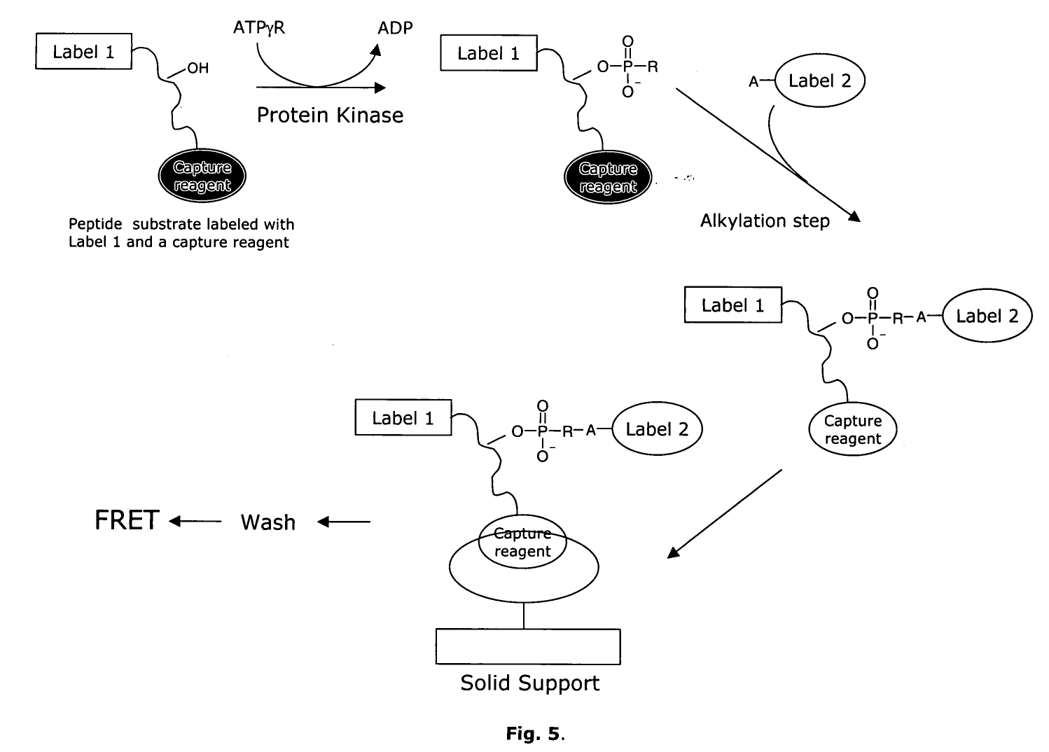

Shown in FIG. 5 is a modification of the method for a discontinuous assay system to allow for wash steps. In this modification the substrate, in this case a peptide, is labeled in addition to label 1 with a capture reagent that will bind specifically with a solid support (i.e. bead, microplate, flow cell, microchip, etc.) via a molecule that binds tightly to the capture reagent. An example of a capture reagent would be biotin and the solid support may be derivitized with avidin to specifically bind the biotinylated peptide. Once bound to the solid support, excess reagents could be washed away. Washing away the unreacted label 2 would dramatically cut down background fluorescence in the “lights-on” format or reduce the inner-filter effect in the “lights-out” format.

The assays may further be used to test potential kinase or phosphatase inhibitors, using methods known to those of ordinary skill in the art. Individual test compounds, or combinations of compounds, may be included in the kinase or phosphatase reactions at various concentrations to determine whether the compounds inhibit phosphate transfer.

Fluorescence Resonance Energy Transfer, or FRET, involves the transfer of excited state energy from a donor fluorophore to an acceptor fluorophore. FRET may occur when the participating fluorescent labels are very close together (i.e. ≦the Forster distance for ≧50% efficient energy transfer), and the respective transition dipoles are aligned for effective transfer. The efficiency of energy transfer may be used to calculate the distance between the two labels. When the two labels are close together and both are fluorescent, then the fluorescence of the donor label is reduced, and the fluorescence of the acceptor label is increased. Appropriate donor and acceptor pairs have a donor emission spectrum that overlaps with the acceptor absorption spectrum. Higher excitation maximum wavelengths for the donor fluorophore are often used in biological assays, as they involve less background interference. Donors having a large Stokes shift, which permit excitation at a wavelength far below the absorbance wavelength of the acceptors, and donors having a high molar absorbance, are often used in biological assays to increase the sensitivity of the assays. Both the quenching of the donor emission (lights out) or the enhanced emission of the acceptor (lights on) may be assayed. Examples of the use of FRET assays, donor/acceptor pairs, and calculations that may be used to determine binding are known to those of ordinary skill in the art, and may be found in, for example, Lakowicz, J. R. “The Principles of Fluorescence Spectroscopy” (2d ed. 1999), and U.S. Pat. No. 6,203,994. Examples of embodiments of donor-acceptor pairs include, but are not limited to, those in the following table:

| Donor | Acceptor |
|---|---|
| Fluorescein | TAMRA |
| TAMRA | QSY ™-7 |
| EDANS | Fluorescein |
| EDANS | QSY ™-35 |
| EDANS | DABCYL |
| Fluorescein | QSY ™-7 |
| Texas Red | QSY ™-21 |

### EXAMPLES

The following are non-limiting examples of methods of the present invention. Those of ordinary skill in the art may determine, for their own purposes, appropriate substrates, appropriate assay conditions, such as amount and type of buffers, salts, metals, pH, and temperature, as well as methods of validating assays for their own purposes, for example, by conducting time course experiments, and, for example by adding inhibitors. Methods of determining appropriate substrates for the present invention are known to those of ordinary skill in the art, and may, for example, include purchasing substrates from a commercial supplier. One example of a source of peptide substrates is Jerini , A. G. (www.jerini.com, Berlin, Germany). Other sources of catalog peptides include Bachem, Calbiochem, Sigma, and Synpep. Other methods of obtaining substrates include using kinase activation loops and creating pseudosubstrates by creating phosphorylation sites in target peptides. Examples of methods of obtaining peptide substrates may be found in, for example, Songyang, Z, et al., Nature, 373:536-39 (1995); Brinkworth, R., et al., Proc. Natl. Acad. Sci., USA 100:74-79 (2003); and Kemp, B. E., et al., TIBS 19:440-44 (1994).

Definitions

FRET=Fluorescence Resonance Energy Transfer

TAMRA=carboxytetramethylrhodamine

1,5-IAEDANS=5-((((2-iodoacetyl)amino)ethyl)amino) naphthalene-1-sulfonic acid

“Lights-on”=both donor and acceptors are fluorescent

“Lights-off”=the donor is fluorescent and the acceptor is a quencher (i.e. extremely low fluorescence quantum yield).

CTF-peptide=EAIYAAPFAKKK labeled at Lys12 with fluorescein

TAMRA-peptide=EAIYAAPFAKKK labeled at Lys12 with carboxytetramethylrhodamine

Materials

c-Abl peptide=EAIYAAPFAKKK, and was labeled with the indicated fluorescent dye at the ε-amino group of Lys12; CT-TAMRA Abl peptide=EAIYAAPFAKKK-TAMRA; NT-TAMRA Syk peptide=TAMRA-EDDEYEEV-OH; CT-EDANS Kemptide=LRRASLGE-EDANS.

The fluorescein peptide was purchased from BioPeptide Corp. of San Diego at >95% purity.

The tetramethylrhodamine peptide was purchased from SynPep of Dublin Calif. at >95% purity.

QSY®-7 maleimide was purchased from Molecular Probes. Stock solutions were prepared immediately prior to use in DMSO. The concentration of the stocks were spectrophotometrically using ε=84,000 at 560 nm.

1,5-IAEDANS was purchased from Molecular Probes. 10 mM stocks were prepared in DMSO immediately prior to use.

ATPγS was purchased from Sigma and stocks were prepared at 100 mM in water.

c-Abl kinase was expressed and purified from a E. coli expressing a DNA sequence coding for c-Abl.

All other reagents were reagent grade.

Peptides may be obtained by any one of a number of methods that are known to those of ordinary skill in the art, such as by enzymatic cleavage, chemical synthesis, or expression of a recombinantly produced peptide. The peptides may also be purchased from a wide variety of sources. Peptides are often synthesized by t-Boc/Fmoc solid-phase chemistry using automated peptide synthesizers. The present examples present embodiments of peptides that may be used. Any peptides may be used in the methods of the invention, where a thiol-directed method of labeling is used, avoiding peptides that contain cysteine is desirable.

### Example 1

### “Lights-On” Fluorescein 1,5-IAEDANS Test

Each 500 μL reaction contains: 2 μM CTF-peptide, 5 μM ATPγS, 100 mM HEPES, pH 7.5, 10 mM MgCl₂, 10 μg/ml c-Abl enzyme.

A sample is made with buffer in place of the c-Abl enzyme as a negative control. The kinase reaction is started with the addition of the ATPγS and is allowed to proceed for 1.5 hr.

Following the kinase reaction, the alkylation step is started with the additon of 1,5-IAEDANS to a final concentration of 15 μM. The alkylation step is allowed to proceed for 2 hr at room temperature.

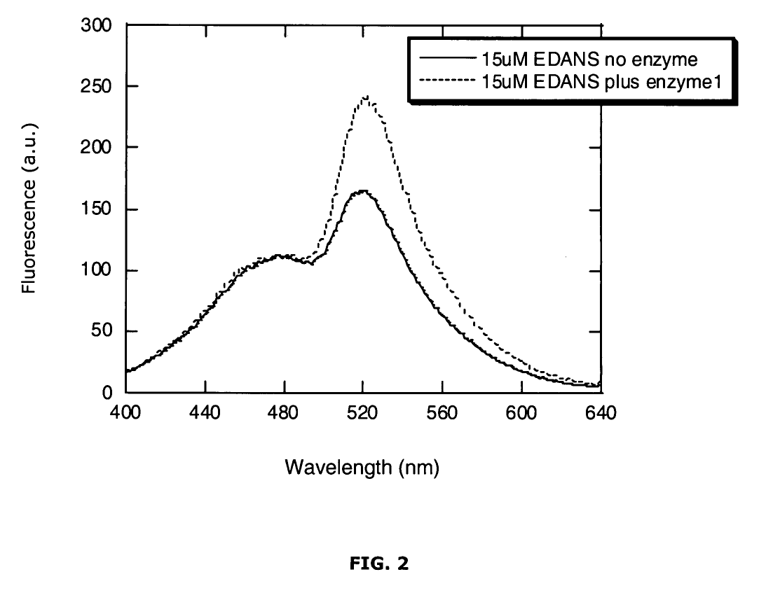

The results of the “lights-on” FRET assay test as described above are shown in FIG. 2.

The assay may be used for other kinases with appropriate peptides including, but not limited to, those presented herein, such as Syk, EGFR, FES, ARG, and PKA.

### Example 2

### “Lights-Out” TAMRA OSY®7 Maleimide Test

Each 400 μL reaction contains: 10 μM TAMRA-peptide, 40 μM ATPγS, 100 mM HEPES, pH 7.5, 10 mM MgCl₂, 10 μg/ml c-Abl enzyme, 0.05% Tween20.

A sample is made with buffer in place of the c-Abl enzyme as a negative control. The kinase reaction is started with the addition of the ATPγS and is allowed to proceed for 1 hr.

Following the kinase reaction, the alkylation step is started with the addition of QSY-7 maleimide to a final concentration of 100 μM. The alkylation step is allowed to proceed for overnight at ambient temperature.

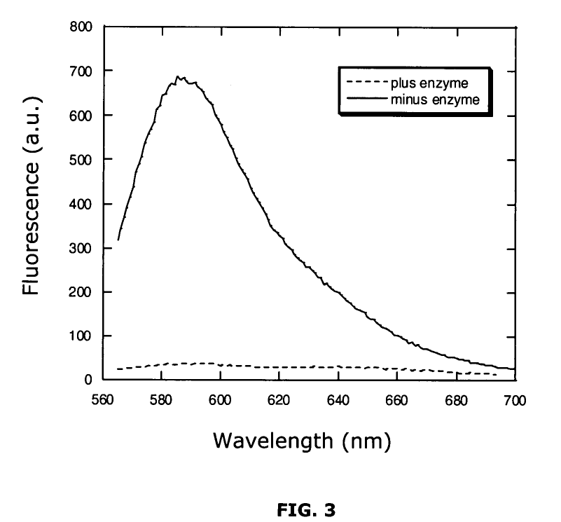

The results of the “lights-out” FRET assay test as described above are shown in FIG. 3.

The activity of the kinase should be directly proportional to the amount of double-labeled peptide. In the lights out format increasing kinase activity will cause a decrease in the measured donor fluorescence. In the lights on assay format increasing kinase activity will produce an increase in measured acceptor fluorescence. Inhibition will interfere with the kinase-dependent production of the double-labeled peptide and thus will cause the opposite effect on the measured fluorescence than the activity will.

### Example 3

### Time Course of Alkylation Experiment

Each 2 mL reaction mix contains 5 μM CT-TAMRA Abl peptide, 10 μM ATPγS 10 μg/mL c-Abl enzyme, 100 mM HEPES, pH 7.5, 1 mM MnCl₂, 40-200 μM QSY 7 malemide, 140 mM β-mercaptoethanol (β-ME, quench).

The kinase reaction consists of c-Abl enzyme, HEPES buffer supplemented with MnCl₂, CT-TAMRA Abl peptide, and ATPγS and is allowed to proceed at room temperature for 1 hour. The reaction is then divided into 5 aliquots and QSY 7 malemide is added to various final concentrations (40-200 μM) to initiate the alkylation reaction. Samples (36 μL) of this reaction are combined with β-ME (4 μL of a 1.4M stock) to quench the alkylation reaction at various time points (0.5-300 minutes). These samples are then diluted 5 fold with buffer and the fluorescence is measured with an excitation of 545 nm and emission of 590 nm.

### Example 4

### Divalent Metal Salt Screens

Each 0.3 mL reaction mix contains 10 μM fluorescently-labeled substrate peptide, 20-60 μM ATPγS, 5-20 μg/mL protein kinase, 100 mM HEPES, pH 7.5, 2 mM each divalent metal salt (water for negative control), 100 μM QSY 7 malemide (for TAMRA-labeled peptides) 400 μM QSY 35 (for EDANS-labeled peptides).

The kinase reaction consists of the indicated protein kinase, HEPES buffer supplemented with 2 mM of the indicated divalent metal salt, fluorescently-labeled peptide, and ATPγS and is allowed to proceed at room temperature for 0.5-1 hour. Along side each reaction an identical reaction supplemented with 50 mM EDTA is run as a negative control for comparison. QSY 7 malemide is added to initiate the alkylation reaction and is allowed to run for >5 hr (typically overnight). These samples are then diluted 3-5 fold with buffer and the fluorescence is measured.

### Example 5

### Time Courses of Kinase Reaction

Each 0.3 mL reaction mix contains 5-10 μM fluorescently-labeled substrate peptide, 10-40 μM ATPγS, indicated concentration of protein kinase (typically 0-20 μg/mL), 100 mM HEPES, pH 7.5, indicated concentration each divalent metal salt, 80-100 μM QSY 7 malemide (for TAMRA-labeled peptides).

The kinase reaction consists of the indicated protein kinase, HEPES buffer supplemented with the indicated concentration of divalent metal salt, fluorescently-labeled peptide. The kinase reaction is initiated by the addition of ATPγS at time t=0. Time points are sampled by combining 36 μL of reaction mix with 4 μL of 0.5M EDTA to quench the reaction. QSY 7 malemide is added to initiate the alkylation reaction and is allowed to run for >5 hr (typically overnight). These samples are then diluted 3-5 fold with buffer and the fluorescence is measured.

Formulation and Administration

Pharmaceutical compositions comprising phosphate transfer inhibitors identified using the methods of the present invention are useful, for example, for modulating protein kinase or phosphatase activity, treatment of conditions mediated by human signal-transduction kinase activity such as, for example, cancer, allergy, asthma, inflammation, and neurodegenerative disorders, as well as disease associated with aberrant cytoskeletal rearrangement, neuronal cell differentiation, and cell cycle progression. While these compounds will typically be used in therapy for human patients, they may also be used in veterinary medicine to treat similar or identical diseases, and may also be used as agents for agricultural use, for example, as herbicides, fungicides, or pesticides.

In therapeutic and/or diagnostic applications, the compounds of the invention can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

The compounds according to the invention are effective over a wide dosage range. For example, in the treatment of adult humans, dosages from 0.01 to 1000 mg, preferably from 0.5 to 100 mg, and more preferably from 1 to 50 mg per day, more preferably from 5 to 40 mg per day may be used. A most preferable dosage is 10 to 30 mg per day. The exact dosage will depend upon the route of administration, the form in which the compound is administered, the subject to be treated, the body weight of the subject to be treated, and the preference and experience of the attending physician. Pharmaceutically acceptable salts are generally well known to those of ordinary skill in the art, may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Pharmaceutically acceptable salts may include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intraperitoneal, intranasal, or intraocular injections.

For injection, the agents of the invention may be formulated in aqueous solutions, for example, in physiologically compatible buffers such as Hank's solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added. The present invention is not to be limited in scope by the exemplified embodiments, which are intended as illustrations of single aspects of the invention. Indeed, various modifications of the invention in addition to those described herein will become apparent to those having skill in the art from the foregoing description and accompanying drawings. Such modifications are intended to fall within the scope of the invention. References cited throughout this application are examples of the level of skill in the art and are hereby incorporated by reference herein in their entirety, whether previously specifically incorporated or not.

I claim:

1. A method of determining the phosphorylating activity of at least one enzyme comprising the steps of:
a. Combining said enzyme with:
i. a phosphorylatable compound, wherein said compound is labeled with an acceptor fluorophore label; and
ii. an ATP analog comprising a reactive species on the γ-phosphate group of said ATP analog;
b. Combining the product of step (a) with a donor fluorophore label, wherein
i. said donor fluorophore label corresponds to the acceptor fluorophore labeling said compound; and wherein
ii. said donor fluorophore label comprises an alkylating moiety that is capable of specifically modifying said reactive species;
c. Measuring the fluorescence resonance energy transfer of the combination of steps (a) and (b); and
d. Using the fluorescence resonance energy transfer of step (c) to determine the phosphorylating activity of the enzyme.

2. The method of claim 1, wherein said enzyme is a kinase.

3. The method of claim 1, wherein said compound is a peptide.

4. The method of claim 3, wherein said peptide comprises an amino acid selected from the group consisting of serine, threonine and tyrosine, and wherein said peptide is capable of being phosphorylated at said amino acid by said enzyme to yield a product.

5. The method of claim 2, wherein said kinase is selected from the group consisting of Abl, Syk, EGFR, FES, ARG, and PKA.

6. A method of determining the phosphorylating activity of an enzyme comprising the steps of:
a. Combining said enzyme with:
i. a phosphorylatable compound, wherein said compound is labeled with a donor fluorophore label; and
ii. an ATP analog comprising a reactive species on the γ-phosphate group of said ATP analog;
b. Combining the product of step (a) with an acceptor fluorophore label, wherein
i. said acceptor fluorophore label corresponds to the donor fluorophore labeling said compound; and wherein
ii. said acceptor fluorophore label comprises an alkylating moiety that is capable of specifically modifying said reactive species;
c. Measuring the fluorescence resonance energy transfer of the combination of steps (a) and (b); and
d. Using the fluorescence resonance energy transfer of step (c) to determine the phosphorylating activity of the enzyme.

7. The method of claim 6, wherein said enzyme is a kinase.

8. The method of claim 6, wherein said compound is a peptide.

9. The method of claim 8, wherein said peptide comprises an amino acid selected from the group consisting of serine, threonine and tyrosine, and wherein said peptide is capable of being phosphorylated at said amino acid by said enzyme to yield a product.

10. The method of claim 7, wherein said kinase is selected from the group consisting of Abl, Syk, EGFR, FES, ARG, and PKA.

11. A method of determining the dephosphorylating activity of an enzyme, comprising the steps of:
a. Combining said enzyme with:
i. a compound, wherein
1. said compound comprises a phosphate group, wherein said phosphate group comprises a reactive species, and wherein
2. said compound is labeled with an acceptor fluorophore label; and
ii. a donor fluorophore label, wherein
1. said donor fluorophore label corresponds to the acceptor fluorophore labeling said compound; and wherein
2. said donor fluorophore label comprises an alkylating moiety that is capable of specifically modifying said reactive species;
b. Measuring the fluorescence resonance energy transfer of the combination of step (a); and
c. Using the fluorescence resonance energy transfer of step (b) to determine the dephosphorylating activity of the test enzyme.

12. The method of claim 19, wherein said enzyme is a kinase.

13. The method of claim 19, wherein said compound is a peptide.

14. The method of claim 21, wherein said peptide comprises an amino acid selected from the group consisting of serine, threonine and tyrosine, and wherein said peptide is capable of being phosphorylated at said amino acid by said enzyme to yield a product.

15. The method of claim 11, wherein said kinase is selected from the group consisting of Abl, Syk, EGFR, FES, ARG, and PKA.

16. A method of determining the dephosphorylating activity of an enzyme, comprising the steps of:
a. Combining said enzyme with:
i. a compound, wherein
1. said compound comprises a phosphate group, wherein said phosphate group comprises a reactive species, and wherein
2. said compound is labeled with a donor fluorophore label; and
ii. an acceptor fluorophore label, wherein
1. said acceptor fluorophore label corresponds to the donor fluorophore labeling said compound, and wherein
2. said acceptor fluorophore label comprises an alkylating moiety that is capable of specifically modifying said reactive species;
b. Measuring the fluorescence resonance energy transfer of the combination of step (a); and
c. Using the fluorescence resonance energy transfer of step (b) to determine the dephosphorylating activity of the test enzyme.

17. The method of claim 24, wherein said enzyme is a kinase.

18. The method of claim 24, wherein said compound is a peptide.

19. The method of claim 26, wherein said peptide comprises an amino acid selected from the group consisting of serine, threonine and tyrosine, and wherein said peptide is capable of being phosphorylated at said amino acid by said enzyme to yield a product.

20. The method of claim 16, wherein said kinase is selected from the group consisting of Abl, Syk, EGFR, FES, ARG, and PKA.

21. The method of any of claims 1-10, wherein said ATP analog comprises ATP-γS.

22. The method of any of claims 11-20, wherein said reactive species is sulfur.

23. The method of any of claims 1-5, and 11-15 wherein said acceptor fluorophore label comprises fluorescein, and said donor fluorophore label comprising an alkylating moiety is 1,5-IAEDANS.

24. The method of any of claims 3-5 and 13-15, wherein
a. Said peptide comprises EAIYAAPFAKKK, comprising said acceptor label at Lys12;
b. Said acceptor fluorophore label comprises fluorescein, and wherein
c. Said donor fluorophore label comprising an alkylating moiety is 1,5-IAEDANS.

25. The method of any of claims 6-10 and 16-20 wherein said donor fluorophore label comprises carboxytetramethylrhodamine, and said acceptor fluorophore label comprising an alkylating moiety is QSY-7 maleimide.

26. The method of any of claims 7-10 and 18-20 wherein
a. said peptide comprises EAIYAAPFAKKK, comprising said donor label at Lys12
b. said donor fluorophore label comprises carboxytetramethylrhodamine, and wherein
c. said acceptor fluorophore label comprising an alkylating moiety is QSY-7 maleimide.

27. An assay system or kit, comprising the following reagents
a. A phosphorylatable compound, wherein said compound is labeled with an acceptor fluorophore label;
b. An ATP analog comprising a reactive species on the γ-phosphate group of said ATP analog;
c. A donor fluorophore label, wherein
i. said donor fluorophore label corresponds to the acceptor fluorophore labeling said compound; and wherein
ii. said donor fluorophore label comprises an alkylating moiety that is capable of specifically modifying said reactive species.

28. An assay system or kit, comprising the following reagents
a. A phosphorylatable compound, wherein said compound is labeled with a donor fluorophore label;
b. An ATP analog comprising a reactive species on the γ-phosphate group of said ATP analog;
c. An acceptor fluorophore label, wherein
i. Said acceptor fluorophore label corresponds to the donor fluorophore labeling said compound; and wherein
ii. said acceptor fluorophore label comprises an alkylating moiety that is capable of specifically modifying said reactive species.

29. The assay system of claim 27 or 28 wherein said compound is a peptide.

30. The assay system of claim 29, wherein said peptide comprises an amino acid selected from the group consisting of serine, threonine and tyrosine, and wherein said peptide is capable of being phosphorylated at said amino acid by said enzyme to yield a product.

31. The assay system of claim 27, wherein said acceptor fluorophore label comprises fluorescein, and said donor fluorophore label comprising an alkylating moiety is 1,5-IAEDANS.

32. The assay system of claim 31, wherein said compound is a peptide.

33. The assay system of claim 32, wherein said peptide comprises an amino acid selected from the group consisting of serine, threonine and tyrosine, and wherein said peptide is capable of being phosphorylated at said amino acid by said enzyme to yield a product.

34. The assay system of claim 33, wherein said peptide comprises EAIYAAPFAKKK, comprising said acceptor label at Lys12;

35. The assay system of claim 28, wherein said donor fluorophore label comprises carboxytetramethylrhodamine, and said acceptor fluorophore label comprising an alkylating moiety is QSY-7 maleimide.

36. The assay system of claim 35, wherein said compound is a peptide.

37. The assay system of claim 36, wherein said peptide comprises an amino acid selected from the group consisting of serine, threonine and tyrosine, and wherein said peptide is capable of being phosphorylated at said amino acid by said enzyme to yield a product.

38. The assay system of claim 37, wherein said peptide comprises EAIYAAPFAKKK, comprising said donor label at Lys12

39. The assay system of claim 27 or 28, wherein each of said reagents is in a separate container.

40. The assay system of claim 39, wherein said containers are enclosed in a package, which package further includes instructions for use of said reagents.

41. The assay system of claim 27 or 28, further comprising a microtray.

42. The method of claim 27 or 28, wherein said ATP analog comprises ATP-γS.
